(19) United States
(12) Patent Application Publication
(10) Pub. No.: US 2007/0093813 A1
(43) Pub. Date: Apr. 26, 2007

(54) Dynamic spinal stabilizer

(76) Inventors: Ronald Callahan II, Naugatuck, CT (US); Ernest Corrao, Bethel, CT (US); Stephen Maguire, Shelton, CT (US); Stephen Santangelo, New Hartford, CT (US)

Correspondence Address: McCarter & English, LLP;Financial Centre, Suite 304A, 695 East Main Street, Stamford, CT, 06901-2138, US

(21) Appl. No.: 11/247,450

(22) Filed: Oct. 11, 2005

(51) Int. Cl. A61F 2/30

(52) U.S. Cl. 606/61

(57) ABSTRACT

An elongated member forming a spinal support rod is implantable adjacent the spine of a patient and includes an axial span or spans for spanning respective spinal levels to promote efficacious spinal support/stabilization. The axial span has an axially articulable geometry, and manifests an angulation mechanism along one or more transverse directions of at least seven degrees across a given spinal level. The angulation mechanism may be associated with joints between structural elements assembled in serial along the axial span, or via a common connection between such structural elements and a restraining element. Rotation between such structural elements can be global. The axial span may have a rod-like profile of a diameter similar to conventional spinal support rods used for lumbar spinal fusion, and provides for use across multiple spinal levels and with multiple adjustable attachment points for associated spine attachment devices to accommodate different patient anatomies.

### BACKGROUND

1. Technical Field

The present disclosure relates to devices, systems and methods for spinal stabilization. More particularly, the present disclosure relates to devices, systems and methods for providing dynamic stabilization to the spine via the use of elongated members spanning one or more spinal levels.

2. Background Art

Each year, over 200,000 patients undergo lumbar fusion surgery in the United States. While fusion is a well-established procedure that is effective about seventy percent of the time, there are consequences even to successful fusion procedures, including a reduced range of motion and an increased load transfer to adjacent levels of the spine, which may accelerate degeneration at those levels. Further, a significant number of back-pain patients, estimated to exceed seven million in the U.S., simply endure chronic low-back pain, rather than risk procedures that may not be appropriate or effective in alleviating their symptoms.

New treatment modalities, collectively called motion preservation devices, are currently being developed to address these limitations. Some promising therapies are in the form of nucleus, disc or facet replacements. Other motion preservation devices provide dynamic internal stabilization of the injured and/or degenerated spine, e.g., the Dynesis stabilization system (Zimmer, Inc.; Warsaw, Ind.) and the Graf Ligament. A major goal of this concept is the stabilization of the spine to prevent pain while preserving near normal spinal function.

In general, while great strides are currently being made in the development of motion preservation devices, the use of such devices is not yet widespread. One reason that this is so is the experimental nature of most such devices. For example, to the extent that a given motion device diverges, whether structurally or in its method of use or implementation, from well-established existing procedures such as lumbar fusion surgery, considerable experimentation and/or testing is often necessary before such a device is given official approval by governmental regulators, and/or is accepted by the medical community as a safe and efficacious surgical option.

With the foregoing in mind, those skilled in the art will understand that a need exists for spinal stabilization devices, systems and methods that preserve spinal motion while at the same time exhibiting sufficient similarity to well-established existing spinal stabilization devices, systems and methods so as encourage quick adoption/approval of the new technology. These and other needs are satisfied by the disclosed devices, systems and methods that include elongated members for implantation across one or more levels of the spine.

### SUMMARY OF THE PRESENT DISCLOSURE

According to the present disclosure, advantageous devices, systems, kits for assembly, and methods for dynamic spinal stabilization are provided. According to exemplary embodiments of the present disclosure, the disclosed devices, systems and methods include an elongated member, e.g., a spinal support rod, that is configured and dimensioned for implantation adjacent the spine of a patient so as to promote efficacious spinal stabilization. The disclosed elongated member is axially articulable and/or manifests angulation means along at least one transverse direction, and is attachable to the spine of a patient via conventional spine attachment hardware, e.g., using pedicle screws, hooks, plates, stems or like apparatus.

According to exemplary embodiments of the present disclosure, the elongated member includes an axial span that extends in an axial direction across at least one spinal level to promote efficacious spinal stabilization thereacross, and has an axially articulable geometry. In some such embodiments, angulation means is manifested in the axial span along at least one transverse direction. Such angulation means can have an extent of at least about five degrees, and/or at least about seven degrees. In some such embodiments, angulation means is manifested in the axial span along at least two transverse directions, and/or global angulation means is manifested therein along transverse directions. In some such embodiments, the axial span is substantially rigid as against axial forces arrayed in compression and/or tension. In some such embodiments, the axial span has a rod-like profile and is adapted to be coupled to the spine of the patient via attachment to conventional spine attachment devices configured for coupling conventional support rods, such as solid, relatively inflexible spinal support rods used in conjunction with spinal fusion assemblies, to the spine. Such rod-like profile can include a diameter in a range from about 5.5 mm to about 6.35 mm, although alternative dimensions and dimensional ranges may be employed, and the axial span can be adapted to permit mounting structures (e.g., pedicle screws, hooks, plates, stems and the like) to be attached to the elongated member at multiple points along the length of the axial span so as to accommodate a range of different patient anatomies and spinal level heights.

Further, in some such embodiments, the elongated member is configured and dimensioned for implantation adjacent the spine such that at least two axial spans of the elongated members extend across respective spinal levels of the spine to promote respective efficacious spinal stabilization thereacross. Both such axial spans are axially articulable.

Some such embodiments of the elongated member also include a plurality of structural elements disposed in series along the axial direction and rotatable relative to each other. Joints can be formed between pairs of adjacent structural elements to permit relative rotation therebetween along respective transverse directions, and such joints can be equipped with stops so as to limit such relative rotation to a predefined extent. Such joints can further permit global rotation between pairs of adjacent structural elements to permit relative rotation along any and/or all transverse directions. Such elongated members can further include a restraining element extending the length of the axial span, wherein the structural elements are coupled to each other via common connections to the restraining element such that relative rotation between and among the structural elements is limited to a predefined, cumulative extent. In such elongated members including a restraining element, the structural elements can render the axial span substantially rigid as against axial forces arrayed in compression, and/or the restraining element renders the axial span substantially rigid as against axial forces arrayed in tension. The restraining element can include a laterally flexible rod along which the structural elements are mounted, and a pair of end caps between which the structural elements are confined. Such laterally flexible rod can be made of a superelastic material, and/or a titanium alloy.

According to further exemplary embodiments of the present disclosure, a surgically implantable spinal support rod is provided that has an axial span that extends in an axial direction so as to span at least one spinal level, wherein the axial span manifests angulation means along at least one transverse direction, and/or manifests global angulation means along transverse directions. In some such embodiments, the axial span has an axially articulable geometry, and the angulation means is a manifestation of such geometry. Some such embodiments of the spinal support rod also include a plurality of structural elements disposed in series along the axial direction and rotatable relative to each other. Joints can be formed between pairs of adjacent structural elements to permit relative rotation therebetween along respective transverse directions. Such joints can further permit global rotation between pairs of adjacent structural elements to permit relative rotation along any and/or all transverse directions. Such spinal support rods can further include a restraining element extending the length of the axial span, wherein the structural elements are coupled to each other via common connections to the restraining element such that relative rotation between and among the structural elements is limited to a predefined, cumulative extent.

In accordance with still further embodiments of the present disclosure, a kit for assembling a dynamic spinal support system is provided. Such kit includes a spinal support rod that has an axial span extending in an axial direction so as to span at least one spinal level, and manifesting angulation means along at least one transverse direction. Such kit also includes a plurality of spine attachment devices respectively attachable to the axial span so as to couple the spinal support rod to the spine of a patient across the spinal level. In some such embodiments, the axial span includes an axially articulable geometry, and the angulation means is a manifestation of such geometry. In some other such embodiments, at least one of the spine attachment devices includes a pedicle screw, hook, mounting plate, stem or the like.

The elongated elements/spinal support rods of the present disclosure, and/or the spinal stabilization devices/systems of the present disclosure incorporating such elongated elements/spinal support rods, advantageously include one or more of the following structural and/or functional attributes:

Spine surgery patients whose conditions indicate that they would benefit from retaining at least some spinal motion in flexion, extension, and/or axial rotation may be fitted with a dynamic spinal stabilization device/system as disclosed herein rather than undergo procedures involving substantial immobilization as between adjacent vertebrae;

The elongated members/spinal support rods in accordance with the present disclosure are compatible (e.g., by virtue of standard diameter sizing, substantial dimensional/diametrical stability, and/or rigidity in axial tension and axial compression, etc.) with most rod attachment hardware presently being implanted in conjunction with lumbar fusion surgery, enhancing the likelihood of quick adoption by the medical community and/or governmental regulatory approval;

The angulation means arising from the axially articulable geometries of the elongated members/spinal support rods disclosed herein results in such members/rods offering little to no resistance to spinal bending to a certain (e.g., predetermined) extent, while providing substantial support/stabilization to the spine (e.g., comparable to solid spinal support bars) when fully deflected and/or positioned at the outer extents of their respective angulation/articulation ranges;

The elongated members/spinal support rods disclosed herein are adaptable to pedicle screw attachment or other attachment structures (e.g., hooks, plates, stems and the like), can be used across one or more spinal levels; manifest at least approximately seven degrees of angulation/articulation with respect to spinal extension and spinal flexion as between adjacent spinal vertebrae, and allow for adjustable attachment points along their axial lengths to accommodate differing patient anatomies.

Advantageous spine stabilization devices, systems, kits for assembling such devices or systems, and methods may incorporate one or more of the foregoing structural or functional attributes. Thus, it is contemplated that a system, device, kit and/or method may utilize only one of the advantageous structures/functions set forth above, or all of the foregoing structures/functions, without departing from the spirit or scope of the present disclosure. Stated differently, each of the structures and functions described herein is believed to offer benefits, e.g., clinical advantages to clinicians or patients, whether used alone or in combination with others of the disclosed structures/functions.

Additional advantageous features and functions associated with the devices, systems, kits and methods of the present disclosure will be apparent to persons skilled in the art from the detailed description which follows, particularly when read in conjunction with the figures appended hereto. Such additional features and functions, including the structural and mechanistic characteristics associated therewith, are expressly encompassed within the scope of the present invention.

### BRIEF DESCRIPTION OF THE DRAWINGS

To assist those of ordinary skill in the art in making and using the disclosed devices, systems and methods, reference is made to the appended figures, in which:

FIGS. 1, 2 and 3 are respective side, top, and end views of a dynamic spinal stabilization device/system implanted into the spine of a patient, in accordance with a first embodiment of the present disclosure;

FIG. 4 is a downward perspective view of an elongated member of the spinal stabilization device/system of FIGS. 1-3;

FIG. 5 is a side illustration of the elongated member of FIG. 4, shown in a partial cutaway view;

FIG. 6 is a side illustration of the spinal stabilization device/system of FIGS. 1-3, wherein the patient is in spinal flexion;

FIG. 7 is a side illustration of the spinal stabilization device/system of FIGS. 1-3, wherein the patient is in spinal extension;

FIGS. 8 and 9 are top views of the spinal stabilization device/system of FIGS. 1-3, wherein the spine of the patient is bending to the left, and to the right, respectively;

FIGS. 10 and 11 are end views of the spinal stabilization device/system of FIGS. 1-3, wherein the spine of the patient is twisting to the right, and to the left, respectively;

FIGS. 12 and 13 are cross-sectional detail views of structural elements of the elongated member of FIGS. 4 and 5 in different states of rotation with respect to each other along a transverse direction coinciding with the plane of the cross-section, illustrating angulation along such transverse direction that is manifested by the axially articulable geometry of the elongated member;

FIG. 14 is a downward perspective view of an elongated member in accordance with a first modification of the spinal stabilization device/system illustrated in FIGS. 1-11;

FIG. 15 is a cross-sectional side illustration of the elongated member of FIG. 14;

FIGS. 16 and 17 are cross-sectional detail views of structural elements of the elongated member of FIGS. 14 and 15 in different states of rotation with respect to each other along a transverse direction coinciding with the plane of the cross-section, illustrating angulation along such transverse direction that is manifested by the axially articulable geometry of the elongated member;

FIG. 18 is a downward perspective view of an elongated member in accordance with a second modification of the spinal stabilization device/system illustrated in FIGS. 1-11;

FIG. 19 is a partial side illustration of the elongated member of FIG. 18, shown in a partial cutaway view; and

FIGS. 20 and 21 are cross-sectional detail views of longitudinal structural elements of the elongated member of FIGS. 18 and 19 in different states of rotation with respect to each other along a transverse direction coinciding with the plane of the cross-section, illustrating angulation along such transverse direction that is manifested by the axially articulable geometry of the elongated member.

### DESCRIPTION OF EXEMPLARY EMBODIMENTS

The present disclosure provides advantageous devices, systems and methods for providing dynamic spinal stabilization. More particularly, the present disclosure provides elongated members and/or spinal support rods that are suitable for surgical implantation across one or more spinal levels for purposes of support and stabilization in flexion, extension, and/or axial rotation, and that include an axially articulable geometry and/or angulation means along transverse directions so as to permit the patient at least some range of motion in spinal flexion, extension, and/or axial rotation while still being capable of providing efficacious support and/or stabilization to the spine.

The exemplary embodiments disclosed herein are illustrative of the advantageous spinal stabilization devices/systems and surgical implants of the present disclosure, and of methods/techniques for implementation thereof. It should be understood, however, that the disclosed embodiments are merely exemplary of the present invention, which may be embodied in various forms. Therefore, the details disclosed herein with reference to exemplary dynamic stabilization systems and associated methods/techniques of assembly and use are not to be interpreted as limiting, but merely as the basis for teaching one skilled in the art how to make and use the advantageous spinal stabilization systems and alternative surgical implants of the present disclosure.

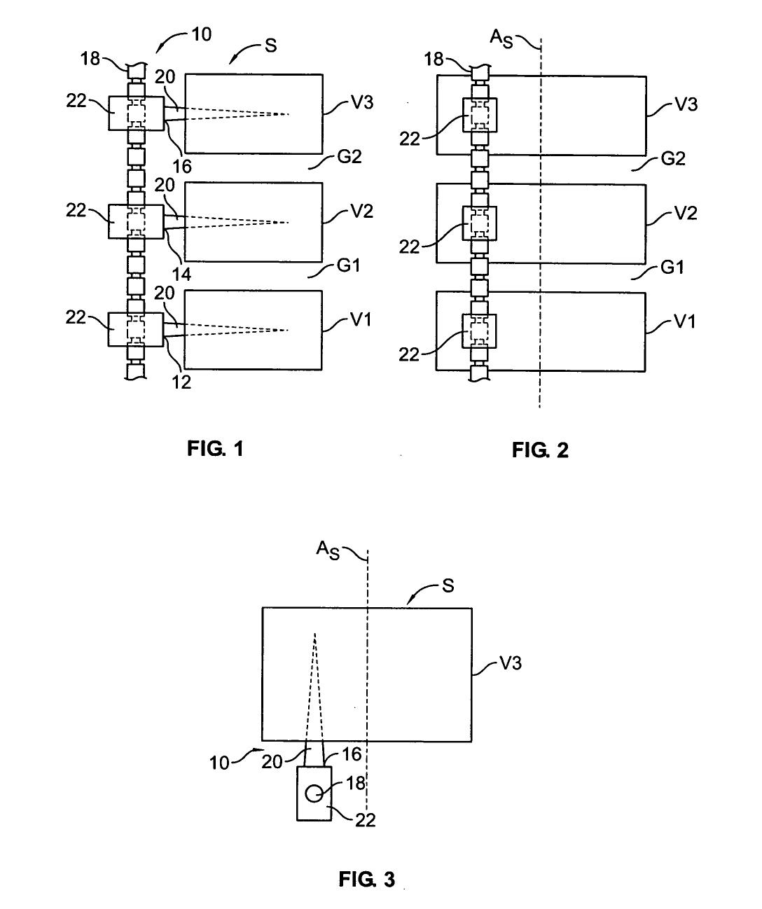

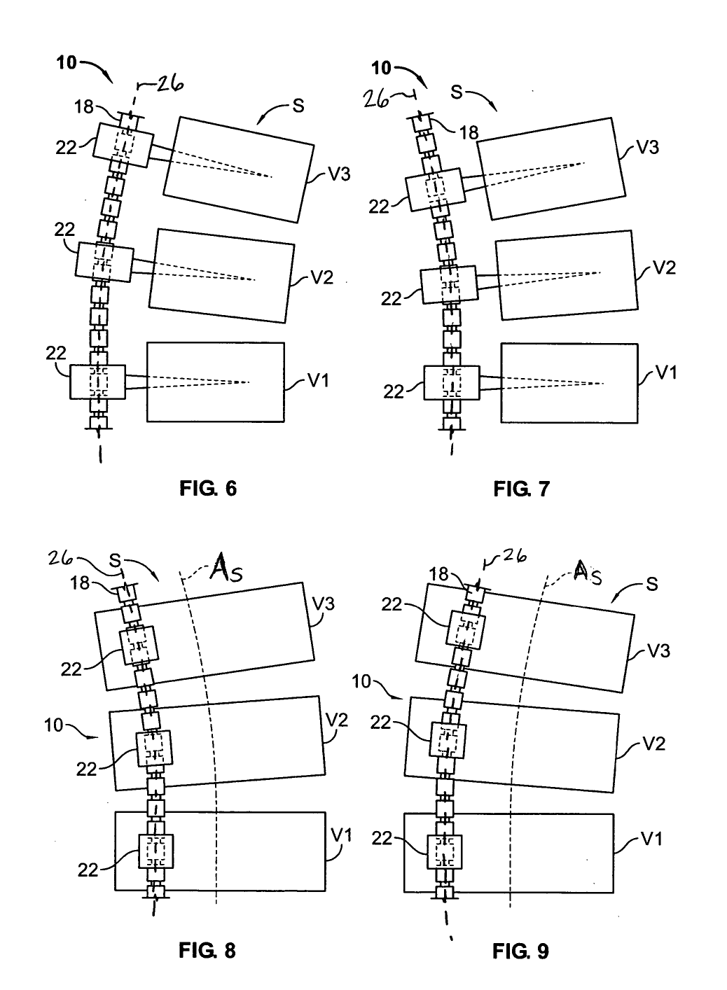

With reference to FIGS. 1-3, a dynamic spinal stabilization system 10 is shown implanted into and/or relative to the spine S of a patient, such spine S being rendered schematically in FIGS. 1-3 (as well as in FIGS. 6-11, the details of which are described more fully hereinbelow) in the form of three adjacent sequential vertebrae V1, V2 and V3 separated by corresponding intervertebral gaps G1 and G2. The dynamic stabilization system 10 is attached to the spine S along one lateral side thereof as defined by a bilateral axis of symmetry Aₛ thereof (another dynamic spine stabilization system 10 (not shown) can be attached to the spine S along the other lateral side thereof as desired and/or as necessary). The spinal stabilization system 10 includes three spine attachment elements 12, 14, 16, and an elongated member 18 spanning all of the vertebrae V1, V2, V3 (e.g., at least insofar as the gaps G1, G2 therebetween).

Each of the spine attachment elements 12, 14, 16 of the spinal stabilization system 10 includes an attachment extension 20 (depicted at least partially schematically) and an attachment member 22 (also depicted at least partially schematically). The spine attachment elements 12, 14, 16 are securely affixed to the respective vertebrae V1, V2, V3 via respective ends of the attachment extensions 20 being embedded within corresponding voids in the tissue of the respective vertebrae V1, V2, V2, and being securely retained therein (i.e., so as to prevent the attachment extensions 20 from being pulled out of their respective voids, or rotated with respect thereto, whether axially or otherwise). The attachment extensions 20 are embedded into and/or retained within their respective vertebral voids via suitable conventional means, such as helical threads and/or a helically-shaped inclined plane formed on the respective attachment extension 20, a biocompatible adhesive, or by other means. The attachment extensions 20 form respective parts of and/or are mounted with respect to, respective pedicle screws of conventional structure and function in accordance with at least some embodiments of the present disclosure. The attachment extensions 20 form parts of other types of structures than that of conventional pedicle screws in accordance with some other embodiments of the present disclosure, e.g., hooks, mounting plates, cemented stems and the like.

The attachment extensions 20 and attachment members 22 of the spine attachment elements 12, 14, 16 are attached or coupled with respect to each other at respective ends of the attachment extensions 20 opposite the ends thereof that are embedded within the tissue of the respective vertebrae V1, V2, V3. Movable joints are advantageously formed at the points where the attachment extensions 20 and the attachment members 22 are attached/coupled. In at least some embodiments of the present disclosure, the ends of the attachment extensions 20 that are attached/coupled with respect to the respective attachment members 22 include respective pedicle screw heads of conventional structure and function. In some other embodiments of the present disclosure, such ends include types of structure other than that of conventional pedicle screw heads (e.g., hooks, mounting plates, stems and the like). The movable joints formed between the attachment extensions 20 and the attachment members 22 may advantageously permit relatively unconstrained relative rotation (e.g., global rotation) therebetween, as well as at least some rotation of each attachment member 22 about an axis defined by the corresponding attachment extension 20. The structure and function of the movable joints between the attachment extensions 20 and the attachment members 22 of the respective spine attachment elements 12, 14, 16 will be described in greater detail hereinafter.

The attachment members 22 of the spine attachment elements 12, 14, 16 are generally configured and dimensioned so as to be operatively coupled to known spinal support rods (not shown) such as spinal support rods of conventional structure and having a standard diameter (e.g., from about 5.5 mm to about 6.35 mm, although alternative dimensions and/or dimensional ranges may also be employed) and that are commonly used in connection with lumbar fusion surgery and/or other spinal stabilization procedures. For example, in accordance with some embodiments of the present disclosure, each of the attachment members 22 is configured to couple to a conventional spinal support rod (not shown) so as to prevent relative movement between the attachment members 22 and the rod in a direction transverse (e.g., perpendicular) to the rod's axial direction of extension, and at least one of the attachment members 22 is further adapted to prevent relative movement between such attachment member 22 and the rod along the rod's axial direction of extension. The particular structures and characteristic functions of the attachment members 22 of the spine attachment elements 12, 14, 16 are discussed in greater detail hereinafter.

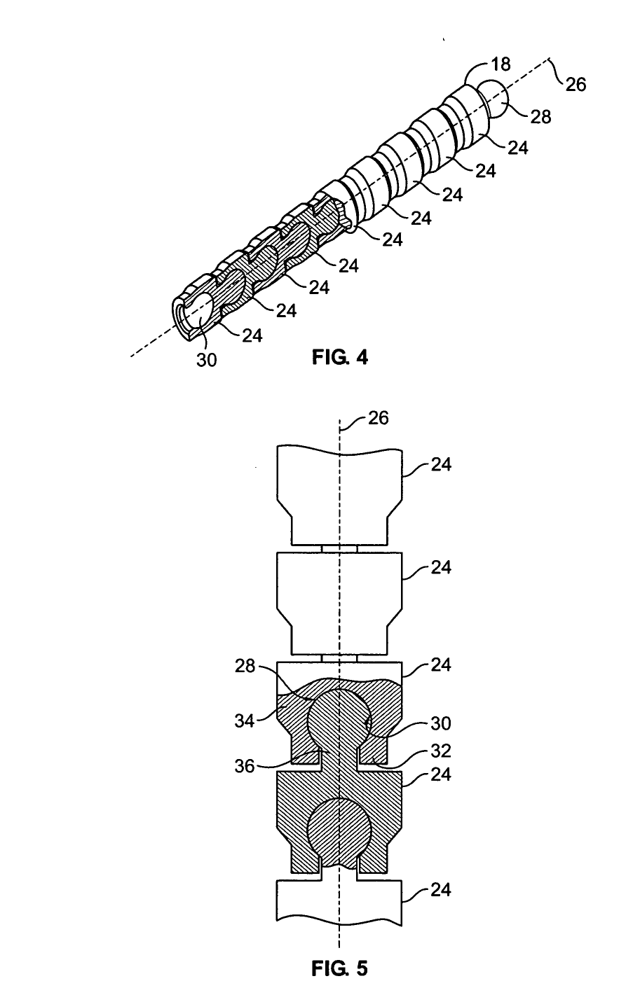

Referring now to FIGS. 4 and 5, the exemplary elongated member 18 of the spinal stabilization system 10 (FIG. 1) is an axially articulable rod made of structural elements 24 that are assembled together in series, and that are capable of rotating relative to each other. More particularly, the serially-arranged structural elements 24 define an axial direction 26 of extension of the elongated member 18. The relative rotation between and among the structural elements 24 produces in the elongated member 18 an articulable aspect whereby the elongated member 18 is to a certain extent relatively flexible and/or non-rigid in the transverse or lateral direction relative to the axial direction 26. In this way, the elongated member 18 manifests angulation means which may be characterized by a “free play” effect, such as is characteristic to certain meshed gear systems, drive chains consisting of individual links, etc. In at least some embodiments of the present disclosure, including the embodiment illustrated in FIGS. 4 and 5, each of the structural elements 24 is substantially similar in structure and function to every other structural element 24. More particularly, each structural element 24 includes a male connector 28 and a female receptor 30. Each male connector 28 of the various structural elements 24 is substantially spherically shaped, and has substantially the same outer diameter, and each female receptor 30 of the various structural elements 24 is substantially spherically shaped, and has substantially the same inner diameter. The characteristic inner diameter of the female receptors 30 is of an extent complementary to that of the characteristic outer diameter of the male connectors 28 such that each female receptor 30 is capable of receiving a corresponding male connector 28 and forming a movable joint (e.g., a global joint) therewith between adjacent structural elements 24, thereby permitting rotational motion between such adjacent structural elements 24 in multiple planes.

In at least some embodiments of the present disclosure, adjacent instances of the structural elements 24 are coupled together via a swaging process in which the male connector 28 of one of a pair of adjacent structural elements 24 is inserted into the female receptor 30 of the other of the pair of adjacent structural elements 24, and an end portion 32 of a main body 34 of the structural element 24 associated with the female receptor 30 is crimped around the male connector 28, and inwardly toward a neck portion 36 of the structural element 24 by which the male connector 28 is connected to the main body 34. Such swaging has the effect of capturing the male connector 28 within the female receptor 30 while providing or permitting at least some rotation of the male connector 28 with respect to the female receptor 30 in multiple planes (e.g., so as to form the global joint between adjacent structural elements 24, as described hereinabove).

The main bodies 34 of the structural elements 24 of the elongated member 18 are generally substantially cylindrically shaped, and exhibit a common outer diameter. In exemplary embodiments of the present disclosure, the outer diameter may be consistent with that of conventional spinal stabilization rods (e.g., having an extent in a range of from about 5.5 mm to about 6.35 mm) such that the elongated member 18 is compatible with hardware designed to couple to conventional spinal stabilization rods and associated anatomical features and criteria, although alternative dimensions and/or dimensional ranges may also be employed according to the present disclosure. Accordingly, and referring again to FIGS. 1-3, the elongated member 18 is compatible with the spine attachment elements 12, 14, 16. More particularly, the elongated member 18 is coupled to the attachment members 22 of the spine attachment elements 12, 14, 16 such that transverse movement of the elongated member 18 relative to the respective attachment members 22 is substantially limited and/or prevented. This is consistent with the support and stabilization function (described in greater detail hereinafter) of the elongated member 18 with respect to the spine S.

With respect to at least one of the attachment members 22, the elongated member 18 is coupled thereto such that motion/translation of the elongated member 18 in the axial direction 26 (FIG. 5) relative to such attachment member(s) 22 is substantially limited and/or prevented. This ensures that the elongated member 18 is prevented from freely and/or uncontrollably moving/translating in the axial direction 26 with respect to the spine attachment elements 12, 14, 16 in the context of the overall spinal stabilization system 10. Moreover, in accordance with the embodiment of the present disclosure illustrated in FIGS. 1-5, the global joints formed between the attachment members 22 and the attachment extensions 20 of the respective spine attachment elements 12, 14, 16 allow the attachment members 22 to rotate to some degree along with the elongated member 18 relative to the spine S. The significance of such flexibility in the elongated member 18, and of the other aspects of the connection between the elongated member 18 and the spine attachment elements 12, 14, 16 mentioned immediately hereinabove, is described more fully hereinafter.

The elongated member 18 is also similar to conventional spinal stabilization rods in that the structural elements 24 thereof, and, particularly, the main bodies 34 of the structural elements 24, are substantially dimensionally stable in the radial direction (e.g., transversely relative to the axial direction 26). Accordingly, the elongated member 18 is capable of withstanding radially-directed compressive forces imposed by any and/or all of the attachment members 22 either during the process of implanting the elongated member 18 along the spine S (e.g., in response to any and/or all clamping forces imposed by any attachment member 22 on the elongated member 18), or during in situ use of the spinal stabilization system 10 (the details of the latter being described more fully hereinafter). In accordance with some embodiments of the present disclosure, the structural elements 24 of the elongated member 18 are made from a biocompatible metallic structural material, such as a titanium or stainless steel alloy. Further with respect to such embodiments, the material and structural aspects of the elongated member 18 described hereinabove render the elongated member 18 substantially rigid in axial tension, as well as substantially incompressible and buckle-resistant when subjected to axially-directed compression forces.

In operation, e.g., when incorporated in the spinal stabilization system 10 adjacent the spine S of a patient as described hereinabove, the elongated member 18 is capable of supporting the spine S in any one or more, or all, of spinal flexion, spinal extension, and spinal axial rotation. As may be seen by comparing FIGS. 1 and 6, the elongated member 18 of spinal stabilization system 10 is sufficiently flexible to deflect, without offering substantial resistance to such motion, from a substantially linear configuration (FIG. 1) to a configuration in which the elongated member 18 includes an anterior bend (FIG. 6). More particularly with respect to FIG. 6, once placed in the geometrical configuration shown therein (i.e., having an anterior bend of such an extent), the elongated member 18 is capable of supporting the vertebrae V1, V2, V3 of the spine S so as to substantially prevent spinal flexion to a greater degree than that which is shown. In accordance with some embodiments of the present disclosure, the elongated member 18 is dimensioned and configured to permit such spinal flexion between adjacent vertebrae (e.g., between vertebrae V1 and V2, or between vertebrae V2 and V3) to an extent of at least approximately seven degrees.

As may be seen by comparing FIGS. 1 and 7, the elongated member 18 of the spinal stabilization system 10 is sufficiently flexible to deflect, without offering substantial resistance to such motion, from a substantially linear configuration (FIG. 1) to a configuration in which the elongated member 18 includes a posterior bend (FIG. 7). More particularly with respect to FIG. 7, once placed in the geometric configuration shown therein, (i.e., having a posterior bend of such an extent), the elongated member 18 is capable of supporting the vertebrae V1, V2, V3 of the spine S so as to substantially prevent spinal extension to a greater degree than that which is shown. In accordance with some embodiments of the present disclosure, the elongated member 18 is dimensioned and configured to permit such spinal extension between adjacent vertebrae (e.g., between vertebrae V1 and V2, or between vertebrae V2 and V3) to an extent of at least approximately seven degrees.

As may be seen by comparing FIG. 2 to FIGS. 8 and 9, respectively, the elongated member 18 of the spinal stabilization system 10 is sufficiently flexible to deflect, without offering substantial resistance to such motion, from a substantially linear configuration (FIG. 2) to a configuration in which the elongated member 18 includes a leftward bend (FIG. 8) or a rightward bend (FIG. 9) as reflected in the respective curves in the axis of symmetry Aₛ of the spine S. More particularly with respect to FIGS. 8 and 9, once placed in the geometric configurations shown therein, (i.e., having a leftward or rightward lateral bend of such an extent), the elongated member 18 is capable of supporting the vertebrae V1, V2, V3 of the spine S so as to substantially prevent spinal lateral bending to a greater degree than that which is shown. In accordance with some embodiments of the present disclosure, the elongated member 18 is dimensioned and configured to permit such spinal lateral bending between adjacent vertebrae (e.g., between vertebrae V1 and V2, or between vertebrae V2 and V3) to an extent of at least approximately seven degrees.

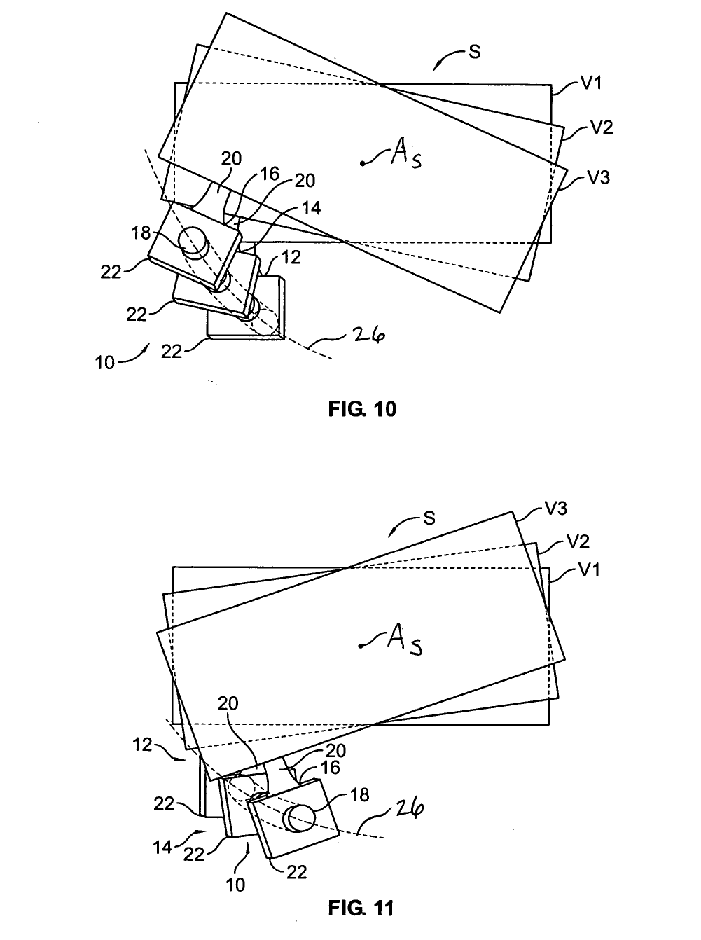

As may be seen by comparing FIG. 3 to FIGS. 10 and 11, respectively, the elongated member 18 of the spinal stabilization system 10 is sufficiently flexible to deflect, without offering substantial resistance to such motion, from a substantially linear configuration (FIG. 3) to a configuration in which the elongated member 18 includes a leftward helical bend (FIG. 10) or a rightward helical bend (FIG. 11) about the axis of symmetry Aₛ of the spine S. More particularly with respect to FIGS. 10 and 11, once placed in the geometrical configurations shown therein, (i.e., having a leftward or rightward helical bend of such an extent), the elongated member 18 is capable of supporting the vertebrae V1, V2, V3 of the spine S so as to substantially prevent spinal twist therein to a greater degree than that which is shown. In accordance with some embodiments of the present disclosure, the elongated member 18 is dimensioned and configured to permit such spinal twist in adjacent vertebrae (e.g., between vertebrae V1 and V2, or between vertebrae V2 and V3). As is particularly evident in the illustrations provided in FIGS. 10 and 11, the global joints between the attachment members 22 and the attachment extensions 20 of the spine attachment elements 12, 14, 16 permit the attachment members 22 ranges of motion relative to the respective attachment extensions 20, and relative to each other, sufficient to track even a complex helical bend, free from undue friction and/or binding.

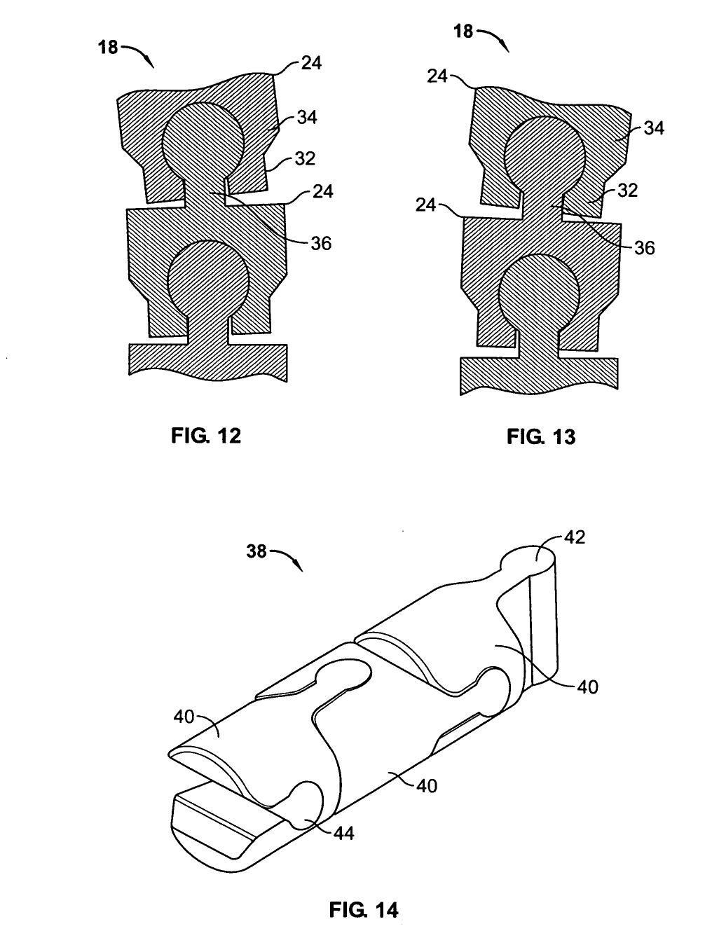

As alluded to hereinabove, the elongated member 18 is laterally and/or transversely flexible and/or non-rigid to a certain extent, but is otherwise substantially laterally and/or transversely rigid. More particularly, and as shown in FIGS. 12 and 13, after a certain extent of relative rotation as between adjacent structural elements 24 of the elongated member 18 associated with the angulation means, the end portion 32 of the main body 34 of one of the adjacent structural elements 24 meets the post 36 of the other of the adjacent structural elements 24, thereby positively preventing further rotation of the adjacent structural elements 24 relative to each other. Such rotation-limiting interactions between adjacent structural elements 24 collectively serve to place a positive limit on the extent of any bend (simple, helical, or otherwise) that may be formed in the elongated member 18 during in situ use. Accordingly, the elongated member 18, and/or the spinal stabilization device 10 (FIG. 1) of which the elongated member 18 forms a part, will impose corresponding limitations on the degree to which the spine S (FIG. 1) that the elongated member 18 is supporting or stabilizing will be permitted to bend or twist.

It should be appreciated that numerous advantages are provided by the elongated member 18 and/or by devices such as the spinal stabilization device 10 that incorporate the elongated member 18 in accordance with the foregoing description to provide dynamic stabilization to the spine of a patient. Spine surgery patients whose conditions indicate that they would benefit from retaining at least some spinal motion in flexion, extension and/or axial rotation may be fitted with the dynamic spinal stabilization device 10 rather than undergo procedures involving substantial immobilization as between adjacent vertebrae. The elongated member 18 (e.g., by virtue of its standard diameter sizing, substantial dimensional stability, and rigidity in tension and/or compression) is compatible with most rod attachment hardware presently being implanted in conjunction with lumbar fusion surgery and other spinal procedures, providing at least some basic similarity between the spinal stabilization device 10 and existing spinal stabilization devices, which similarity is advantageous insofar as it tends to simplify the process of seeking widespread industry acceptance and/or regulatory approval. The elongated member 18 offers little to no resistance to lateral bending to a certain (e.g., predetermined) extent, yet positively prevents lateral bending beyond such certain extent consistent with its spinal support/stabilization function. The elongated member 18 is adaptable to pedicle screw attachment and other mounting apparatus (e.g., hooks, plates, stems and the like), allows for its use across two or more spinal levels, permits at least approximately seven degrees of lateral flexibility in spinal extension and spinal flexion as between adjacent spinal vertebrae, and allows for adjustable pedicle screw attachment points along the elongated member 18 to accommodate differing patient anatomies. Other advantages are also provided.

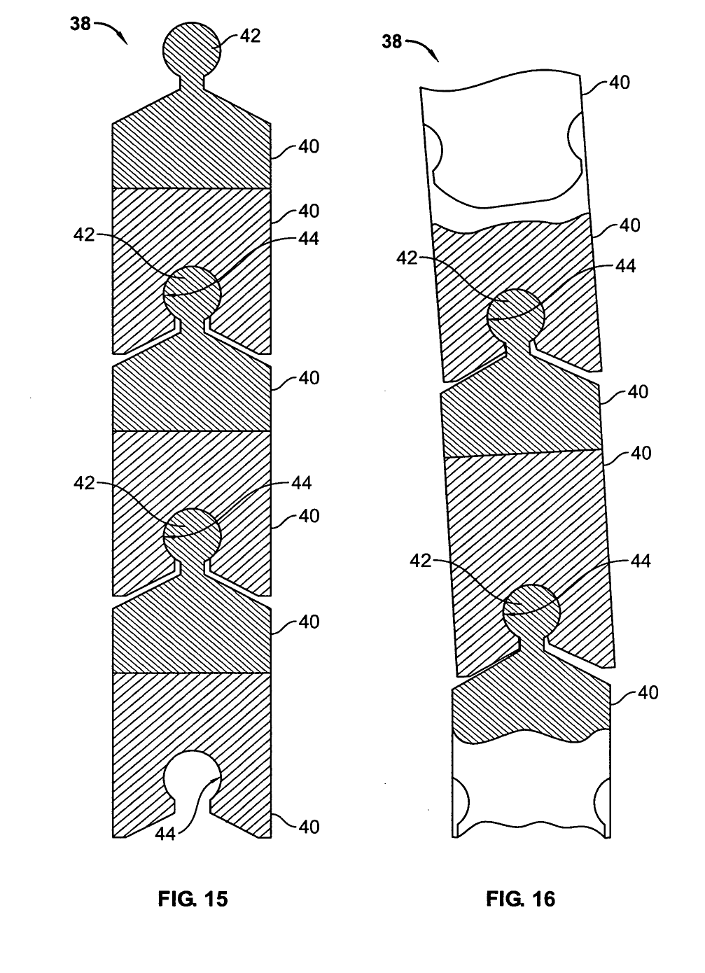

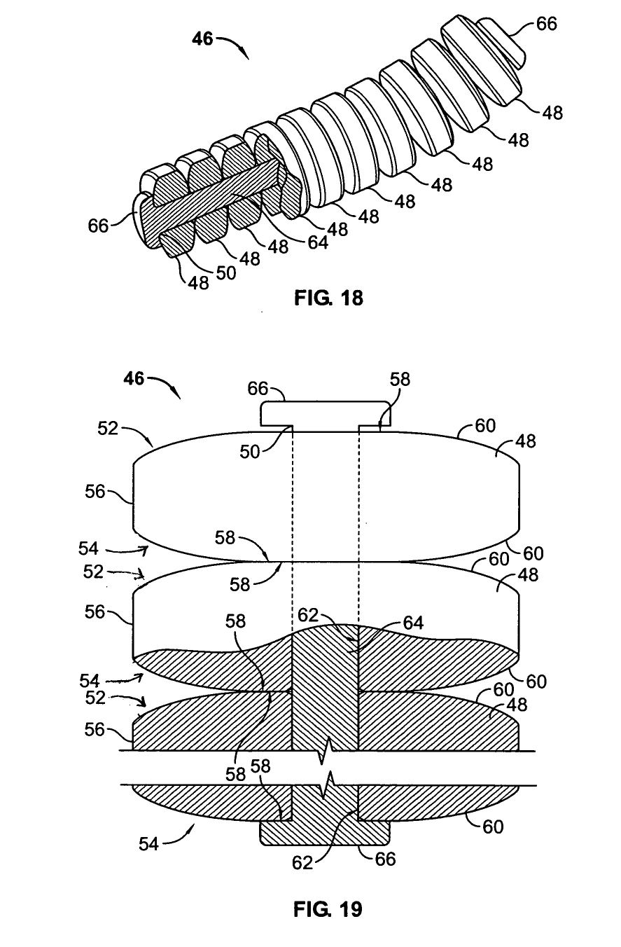

It should also be noted that the elongated member 18, and/or the dynamic spinal stabilization device 10 of which the elongated member 18 forms a part, are subject to numerous modifications and/or variations. For example, the structural elements 24 of the elongated member 18, rather than being interconnected via global joints, can be interconnected in other ways, such as via single-plane rotation joints (see, e.g., FIGS. 14-17 and corresponding description provided hereinbelow), and/or a via a common connection to a third element of structure (see, e.g., FIGS. 18-21 and corresponding description provided hereinbelow), etc. The elongated member 18 can be attached in many different ways to the attachment members 22 of the respective spine attachment elements 12, 14, 16, including embodiments wherein at least one of the attachment members 22 includes an axial hole through which the elongated member 18 either extends freely in the axial direction, or is clamped in place so as to prevent relative axial motion/translation, and embodiments wherein at least one of the attachment members 22 forms a hook (e.g., an incomplete hole) that includes no clamping means and therefore does not limit axial relative motion/translation of the elongated member 18. Many other variations in the spine attachment elements 12, 14, 16 are also possible, including the number of same provided in the context of the spinal stabilization device 10 (e.g., only two, four or more, etc.), as well as the method by which any or all are attached to their respective spinal vertebrae. The elongated member 18 can accordingly be shortened or lengthened (e.g., the number of structural elements 24 can be reduced or increased), so as to be suitable for spanning a single pair of adjacent vertebrae, or more than three adjacent vertebrae. Rather than contacting the actual respective posts 36 to place a limit on relative rotation between adjacent structural elements 24, the end portions 32 of the structural elements 24 can contact surfaces or points along the main bodies 34 of the adjacent structural elements 24.

FIGS. 14-17 illustrate an elongated member 38 that represents a modification to the spinal stabilization device 10 of FIGS. 1-11 in that the elongated member 38 can be substituted for the elongated member 18 (FIGS. 1-13) in at least some circumstances. Referring to FIG. 14, the elongated member 38 is substantially similar in structure and/or function to the elongated member 18 shown and described hereinabove (some such similarities being enumerated below), with exceptions at least insofar as are described hereinbelow. The elongated member 38 includes structural elements 40 which are rotatable relative to each other via corresponding male and female receptors 42, 44 having corresponding respective outer and inner diameters. Rather than being spherical in shape, and therefore accommodating multiplane rotation between the adjacent structural elements in the manner of the elongated member 18, the male and female receptors 42, 44 are cylindrical in shape, and thereby allow rotation in one plane only per pair of connectors 42, 44. Either or both the male or female receptors 42, 44 is swaged and/or indexed, e.g., on at least one end or elsewhere, to prevent dislocation and/or disconnection between the structural elements 40. Adjacent pairs of connectors 42, 44 are rotated ninety degrees relative to each other, and the elongated member 38 consists of many such structural elements 40 (e.g., many more structural elements 40 than are shown in FIG. 14), such that the elongated member 38 is ultimately still capable of bending in any desired direction through varying degrees of cooperation among the differently-oriented pairs of connectors 42, 44 (though perhaps not with as smooth a bending profile as that which can be achieved by the elongated member 18 shown and described hereinabove).

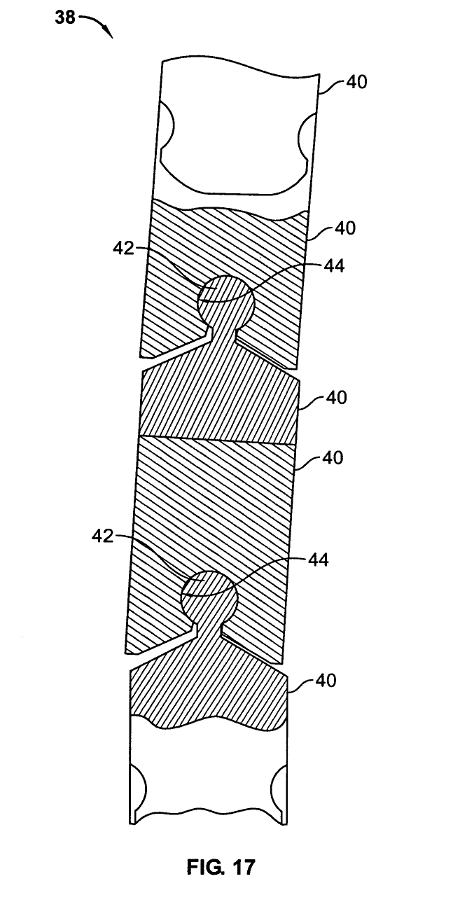

As shown in FIGS. 15-17, when bending of the elongated member 38 takes place solely in the plane of a given pair of connectors 42, 44, two structural elements 40 must rotate in unison (e.g., without the possibility of rotation in the joint they share) relative to two other adjacent structural elements 40, similarly rotationally joined. Similarly to the elongated member 18 shown and described hereinabove, positive limits are placed (see FIGS. 16 and 17) on the degree to which adjacent structural elements 40 can rotate relative to each other within an angulation/articulation range, consistent with the important support and stabilization function of the elongated member 38. The outer diameter and materials of the elongated member 38 are generally similar to the elongated member 18 described hereinabove, providing similar compatibility with existing spine attachment hardware as well as adequate rigidity when the elongated member 38 reaches the end of its range of flexibility and is actively providing spinal support/stabilization.

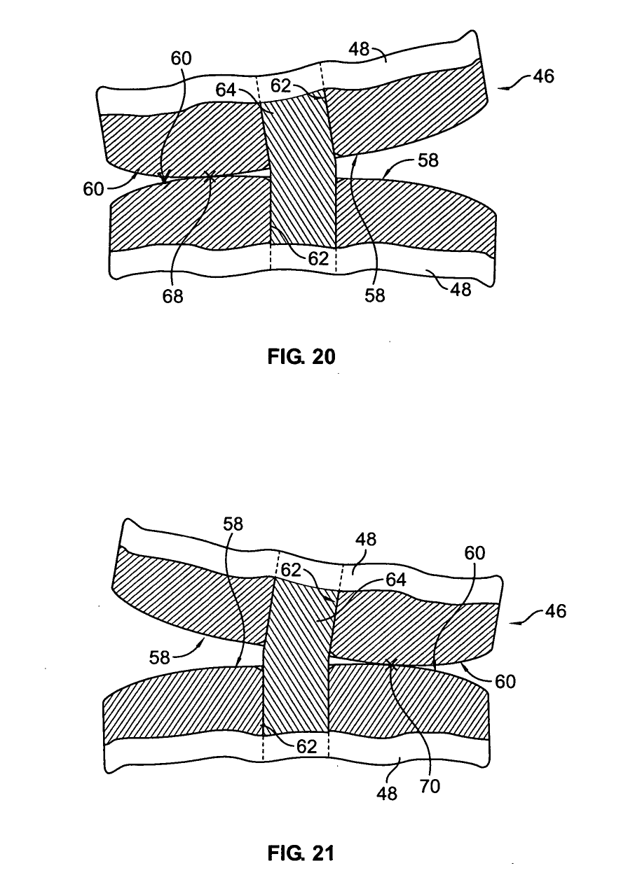

FIGS. 18-21 illustrate an elongated member 46 that represents an alternative modification to the spinal stabilization device 10 of FIGS. 1-11, in that the elongated member 46 can also be substituted for the elongated member 18 (FIGS. 1-13) in at least some circumstances. For example, the elongated member 46 can be utilized as a substitute for the elongated members 18 and 38 in the context of the above-described spinal stabilization device 10 in at least some circumstances, and therefore represents a potential modification of the spinal stabilization device 10. Referring to FIGS. 18 and 19, the elongated member 46 includes a series of structural elements 48 stack mounted along a core element 50. Each structural element 48 has a first side 52, a second side 54 opposite the first side 52, and a peripheral edge surface 56 that is substantially cylindrical, such that the structural element 48 appears substantially circular in shape when viewed from either of the first or second sides 52, 54. Each of the first and second sides 52, 54 of each structural element 48 includes a centrally located planar surface 58 that has a circular outline, and a rounded surface 60 disposed between the circular outline of the planar surface 58 and the peripheral edge surface 56. The planar surfaces 58 of each structural element 48 are oriented parallel to each other and are spaced apart from each other by a distance corresponding to the maximum thickness of the structural element 48. Each structural element 48 further includes a hole 62 that passes between the planar surfaces 58 thereof, is straight and round, and is axially aligned with the peripheral edge surface 56 of the structural element 48.

The rounded surfaces 60 of the structural elements 48 are smoothly tapered relative to the corresponding planar surfaces 58 such that the planar surfaces 58 are substantially tangentially oriented relative to the rounded surfaces where the two surfaces meet. The rounded surfaces 60 of the structural elements 48 are also characterized by a relatively large radius of curvature immediately adjacent thereto such that the profile of the rounded surfaces 60 near the corresponding planar surfaces 58 is that of a shallow curve, and such that the thicknesses of the structural elements 48 at various radial distances from the planar surfaces 58 are generally not significantly less than the maximum thickness thereof between the planar surfaces 58. The radius of curvature of the rounded surfaces 60 of each structural element 48 adjacent the peripheral edge surfaces 56 is relatively small, thereby providing the structural element 48 with a smooth outer profile.

The core element 50 includes a core rod 64 and an end cap 66 at each of two opposite ends of the core rod 64. The core rod 64 may be advantageously fabricated (in whole or in part) from a superelastic material, e.g., a nickel titanium alloy that is relatively inextensible for present purposes (e.g., based on the types and levels of forces to which the core rod 64 can be expected to be exposed in situ, and/or during representative mechanical testing). The core rod 64 is further substantially circular in cross section, extends substantially the entire length of the elongated member 46, and is of a relatively narrow gage (e.g., 2 mm or less) so as to more or less freely permit a considerable degree of lateral flexure in the core rod 64 while remaining safely within the elastic range of the material of the core rod 64 (i.e., without substantial risk of the core rod 64 undergoing plastic/permanent deformation).

The core rod 64 of the core element 50 extends through holes 62 formed in the structural elements 48. The holes 62 of the structural elements 48 are of a common diameter only slightly larger than that of the core rod 64 so as to limit free play of the core rod 64 within the holes 62, and encourage the peripheral edge surfaces 56 of the structural elements 48 to remain substantially aligned with each other along an axial direction of extension of the elongated member 46. This contributes to the overall dimensional stability of the elongated member 46 and/or to the ability of attachment members of corresponding spine attachment elements to interact with and/or connect to the elongated member 46. The end caps 66 are axially affixed to the opposite ends of the core rod 64 adjacent the outermost planar surfaces 58 of the structural elements 48, thereby retaining the structural elements 48 in a mounted configuration along the core element 50. The core rod 64 is of a length that permits a certain (e.g., predefined) amount of slack or free play among the structural elements 48 between the end caps 66, which slack or free play is at its greatest extent when the elongated member 46 is in a straight or unbent configuration (see, e.g., FIG. 19). The functions associated with this aspect of the structure of the elongated member 46 will be explained more fully hereinafter.

Similar to the elongated members 18, 38 shown and described hereinabove, the elongated member 46 can, in at least some circumstances and/or surgical applications, be substituted for a relatively rigid spinal stabilization rod. More particularly, the peripheral surfaces 56 of the structural elements 48 are aligned with each other and are dimensioned so as to exhibit a common outer diameter consistent with that of conventional spinal stabilization rods (e.g., having a range of from about 5.5 mm to about 6.35 mm, although alternative dimensions and/or dimensional ranges may be employed). Accordingly, the elongated member 46 is compatible with hardware designed to couple to conventional spinal stabilization rods, and can therefore be substituted for the elongated member 18 in the spine stabilization device 10 shown and described hereinabove.

In operation, the elongated member 46 is adapted to undergo a certain (e.g., predefined) extent of lateral bending in any/all directions without offering substantial resistance to such lateral bending. The elongated member 46 is further adapted to firmly resist undergoing further lateral bending beyond such certain extent, consistent with the spinal support and/or stabilization function of the elongated member 46. Referring now to FIGS. 20 and 21, initial bending of the elongated member 46 relative to a straight configuration (see FIG. 19) (e.g., as a result of angulation) is driven by spinal movement and involves relative rotation among the structural elements 48 of the elongated member 46 such that adjacent planar surfaces 58 of adjacent pairs of structural elements 48 will tend to separate and rotate away from each other. Such rotation of the structural elements 48 relative to each other necessarily produces elastic bending in the core rod 64, since the core rod 64 is captured within the axial holes 62 of the respective structural elements 48 and must change shape accordingly. Such rotation of the adjacent planar surfaces 58 relative to each produces point contact (indicated in FIGS. 20-21 by reference numerals 68 and 70, respectively) between adjacent rounded surfaces 60 of the structural elements. During such rotation, such point contact serves as a fulcrum/force transmission point between adjacent structural elements 48, such that increased rotation between the structural elements 48 results in increased axial separation between the adjacent planar surfaces 58. Since the rounded surfaces 60 are smoothly tapered to the respective planar surfaces 58, and have shallow profiles adjacent thereto, such point contact 68, 70 arises smoothly and/or without lockup, and the locus of such point contact moves steadily radially outwardly along the rounded surfaces as the extent of rotation between the adjacent structural elements 48 grows. The increased axial separation between the adjacent planar surfaces 58 that is produced thereby tends to take up the aforementioned slack or free play between the end caps 66 (FIG. 19). Once the elongated member 46 has undergone a certain (e.g., predefined) amount of lateral bending (e.g., such certain amount being of lateral bending being associated with significant localized bending at a particular point along the length of the elongated member 46, gradual bending along the entire length of the elongated member 46, a combination thereof, etc.), the slack or free play between the end caps 66 is eliminated. At this point, the outermost sides 52, 54 of the outermost structural elements 48 press steadily axially outward against the end caps 66, which respond by pressing inward on the structural elements 48 with equal and opposite force, and thus preventing any further axial separation as between the adjacent planar surfaces 58 of the structural elements 48. The end caps 66 are braced/coupled together and/or prevented from any further axial separation relative to each other by virtue of the substantial axial inextensibility of the core rod 64 affixed to and extending between the end caps 66. More particularly, while the inherent lateral flexibility of the core rod 64 readily facilitates bending of the elongated member 46 at least to a certain extent, once the elongated member 46 reaches that certain extent of bending, the axial inextensibility of the core rod 64 dominates, and prevents any further bending of the elongated member 46 by positively restricting further rotational movement of the individual structural elements 48 relative to (e.g., axially apart from) each other.

It should be appreciated that numerous advantages are provided by the elongated member 46 and/or by spine stabilization devices (e.g., spine stabilization device 10 shown and described hereinabove) incorporating the elongated member 46. The elongated member 46 offers little to no resistance to lateral bending to a predetermined extent, yet positively prevents lateral bending beyond such predetermined extent consistent with its spinal support/stabilization function. The structural elements 48 feature precisely controllable thicknesses between their respective pairs of planar surfaces 58, smoothly curved rounded surfaces 60 which serve as convenient fulcrums to accommodate the full extent of relative rotation that is permitted between and among the structural elements 48, and dimensionally stable reaction surfaces in the form of peripheral edge surfaces 56 that are configured to interact/cooperate with the attachment members of corresponding spine attachment elements. The core rod 64 of the core element 50 may be made of a superelastic material (e.g., a nickel titanium alloy) such that it exhibits considerable flexibility in lateral bending, while at the same time being substantially axially inextensible for purposes of limiting such lateral bending to a specific (e.g., predetermined) extent. As with the above-described elongated members 18 and 34, the elongated member 46 is adaptable to pedicle screw attachment, allows for its use across two or more spinal levels, permits at least approximately seven degrees of lateral flexibility in spinal extension and spinal flexion as between adjacent spinal vertebrae, and allows for adjustable pedicle screw attachment points along the elongated member 46 to accommodate differing patient anatomies.

It should also be noted that the elongated member 46 can have numerous modifications and/or variations consistent with this embodiment of the present disclosure. The core rod 64 can be made of materials other than superelastic materials, and/or other than metallic materials. The core rod 64 need not necessarily be axially located with respect to the peripheral edge surfaces 56 of the structural elements 48, and can be replaced with and/or supplemented by one or more of a wire-rope cable, a chain, an articulable rod, and/or other structure configured to perform the functions described hereinabove with reference to the core rod 64. The core rod 64 further need not necessarily be circular or even axially or bilaterally symmetrical in cross-sectional shape. The structural elements 48 can be made of metallic or other materials, and it is not specifically necessary that all of the structural elements 48 of the elongated member 46 exhibit the same shape or profile with respect to their respective rounded surfaces 60, and/or the same outer diameter or circular shape as defined by their respective peripheral edge surfaces 56.

It will be understood that the embodiments of the present disclosure are merely exemplary and that a person skilled in the art may make many variations and modifications without departing from the spirit and scope of the invention. All such variations and modifications, including those discussed above, are therefore intended to be included within the scope of the present invention as described by the following claims appended hereto.

1. An elongated member configured and dimensioned for implantation adjacent the spine of a patient such that an axial span of said elongated member extends in an axial direction across at least one spinal level thereof and is adapted to promote efficacious spinal stabilization across said at least one spinal level, said axial span further having an axially articulable geometry.

2. An elongated member according to claim 1, wherein said axially articulable geometry is manifested by angulation means in said axial span along at least one transverse direction.

3. An elongated member according to claim 2, wherein said angulation means has an extent of at least about five degrees.

4. An elongated member according to claim 2, wherein said angulation means has an extent of at least about seven degrees.

5. An elongated member according to claim 1, wherein said axially articulable geometry manifests angulation means in said axial span along at least two transverse directions.

6. An elongated member according to claim 1, wherein said axially articulable geometry manifests global angulation means in said axial span along transverse directions.

7. An elongated member according to claim 1, wherein said axial span is substantially rigid as against axial forces arrayed in compression.

8. An elongated member according to claim 1, wherein said axial span is substantially rigid as against axial forces arrayed in tension.

9. An elongated member according to claim 1, wherein said axial span has a rod-like profile, and is adapted to be coupled to said spine of said patient via attachment to spine attachment devices configured for coupling conventional support rods to said spine.

10. An elongated member according to claim 9, wherein said rod-like profile of said elongated member includes a diameter in a range of from about 5.5 mm to about 6.35 mm.

11. An elongated member according to claim 1, wherein said axial span is adapted to permit mounting apparatus to attach to said elongated member at multiple points along said axial span so as to accommodate a range of different patient anatomies and spinal level heights.

12. An elongated member according to claim 1, wherein said elongated member is configured and dimensioned for implantation adjacent the spine of the patient such that at least two axial spans of said elongated member extend in respective axial directions across respective spinal levels thereof and are respectively adapted to promote efficacious spinal stabilization across said respective spinal levels, each axial span of said at least two axial spans having an axially articulable geometry.

13. An elongated member according to claim 1, wherein said axially articulable geometry includes a plurality of structural elements disposed in series along said axial direction and transversely rotatable relative to each other.

14. An elongated member according to claim 13, wherein said axially articulable geometry further includes a plurality of joints formed between adjacent ones of said plurality of structural elements, each joint of said plurality of joints permitting a pair of adjacent ones of said plurality of structural elements to rotate relative to each other along a respective transverse direction.

15. An elongated member according to claim 14, wherein each joint of said plurality of joints includes a stop so as to substantially limit said respective pair of adjacent ones of said plurality of structural elements to a predefined extent of rotation relative to each other along said respective transverse direction.

16. An elongated member according to claim 13, wherein said axially articulable geometry further includes a plurality of global joints formed between adjacent ones of said plurality of structural elements, each global joint of said plurality of global joints permitting a pair of adjacent ones of said plurality of structural elements to rotate relative to each other along substantially any transverse direction.

17. An elongated member according to claim 13, wherein said axially articulable geometry further includes a restraining element extending along substantially an entire length of said axial span, and wherein said structural elements are coupled to each other via common connections to said restraining element such that relative rotation between and among said structural elements is limited to a predefined cumulative extent.

18. An elongated member according to claim 17, wherein said structural elements render said axial span substantially rigid as against axial forces arrayed in compression.

19. An elongated member according to claim 17, wherein said restraining element renders said axial span substantially rigid as against axial forces arrayed in tension.

20. An elongated member according to claim 17, wherein said restraining element includes a laterally flexible rod along which said structural elements are mounted, and a pair of end caps between which said structural elements are confined.

21. An elongated member according to claim 20, wherein said laterally flexible rod is made of a superelastic material.

22. An elongated member according to claim 20, wherein said laterally flexible rod is made of a titanium alloy.

23. A surgically implantable spinal support rod having an axial span extending in an axial direction so as to span at least one spinal level, said axial span manifesting angulation means along at least one transverse direction.

24. A spinal support rod according to claim 23, wherein said axial span manifests global angulation means along transverse directions.

25. A spinal support rod according to claim 23, wherein said axial span has an axially articulable geometry, and said angulation means is a manifestation of said axially articulable geometry.

26. A spinal support rod according to claim 25, wherein said axially articulable geometry includes a plurality of structural elements disposed in series along said axial direction and transversely rotatable relative to each other.

27. A spinal support rod according to claim 26, wherein said axially articulable geometry further includes a plurality of joints formed between adjacent ones of said plurality of structural elements, each joint of said plurality of joints permitting a pair of adjacent ones of said plurality of structural elements to rotate relative to each other in a respective transverse direction.

28. A spinal support rod according to claim 26, wherein said axially articulable geometry further includes a plurality of global joints formed between adjacent ones of said plurality of structural elements, each joint of said plurality of joints permitting a pair of adjacent ones of said plurality of structural elements to rotate relative to each other along substantially any transverse direction.

29. A spinal support rod according to claim 26, wherein said axially articulable geometry further includes a restraining element extending along substantially an entire length of said axial span, and wherein said structural elements are coupled to each other via common connections to said restraining element such that relative rotation between and among said structural elements is limited to a predefined cumulative extent.

30. A kit for assembling a dynamic spinal support system, comprising:
a spinal support rod having an axial span extending in an axial direction so as to span at least one spinal level, and manifesting angulation means along at least one transverse direction; and
a plurality of spine attachment devices attachable to said axial span so as to couple said spinal support rod to the spine of a patient across said at least one spinal level.

31. A kit for assembling a dynamic spinal support system according to claim 30, wherein said axial span includes an axially articulable geometry, and said angulation means is a manifestation of said axially articulable geometry.

32. A kit for assembling a dynamic spinal support system according to claim 30, wherein at least one of said spine attachment devices is selected from the group consisting of a pedicle screw, a hook, a mounting plate and a stem.
